(19) United States
(12) Patent Application Publication
(10) Pub. No.: US 2023/0389926 A1
(43) Pub. Date: Dec. 7, 2023

(54) BIODEGRADABLE NANOFIBER CONICAL CONDUITS FOR NERVE REPAIR

(71) Applicant: The Johns Hopkins University, Baltimore, MD (US)

(72) Inventors: Hai-Quan Mao, Baltimore, MD (US); Sami Tuffaha, Baltimore, MD (US); Ahmet Hoke, Baltimore, MD (US); Michael James Lan, Baltimore, MD (US); Bruce Peter Enzmann, Baltimore, MD (US); Anson Y. Zhou, Baltimore, MD (US); Alison L. Wong, Baltimore, MD (US); Erica Lee, Baltimore, MD (US); Sai Pinni, Baltimore, MD (US)

(21) Appl. No.: 18/249,186

(22) PCT Filed: Oct. 14, 2021
(86) PCT No.: PCT/US2021/054999

§ 371 Date: Apr. 14, 2023

Related U.S. Application Data
(60) Provisional application No. 63/092,616, filed on Oct. 16, 2020.

(51) Int. Cl. A61B 17/11; A61L 27/52

(52) U.S. Cl. CPC A61B 17/1128; A61B 2017/00893; A61L 27/52

(57) ABSTRACT

Biodegradable nanofiber conical conduits for nerve repair and methods of using same are disclosed. The biodegradable nanofiber conical conduits for nerve repair and methods provide a saturable conduit having a conical shape/geometry including a larger proximal aperture and smaller distal aperture to mechanically guide the regenerating axons across the mismatched repair and thereby prevent axonal escape and neuroma formation. The biodegradable nanofiber conical conduits for nerve repair may include, but are not limited to, a conical conduit that tapers substantially linearly; a conical conduit including a conical concave shape, a conical conduit including a conical convex shape, a conical conduit including proximal and/or distal extensions, a conical conduit including an arrangement of lateral or radial ridges for crimping action, and a conical conduit filled with hydrogel for inhibiting excessive axonal growth.

### TECHNICAL FIELD

The presently disclosed subject matter relates generally to methods of nerve repair and more particularly to biodegradable nanofiber conical conduits for nerve repair and methods of using same.

### BACKGROUND

There are an estimated two million limb amputees living in the U.S. alone and the rate of amputation continues to rise due to the increasing prevalence of diabetes and peripheral vascular disease. Up to 75% of these patients have severe neuroma pain that can be addressed using targeted muscle reinnervation (TMR). Reinnervation is the process of innervating a part of the body that has lost nerve supply (as from injury or disease). Namely, the restoration of function to a denervated body part and especially a muscle by supplying it with nerves through regrowth or grafting. TMR is a surgical procedure used to prevent painful neuromas from occurring, typically at the time of limb amputation, and to treat painful neuromas that have already occurred. TMR also is used to treat neuromas that occur in other clinical scenarios beyond amputation. A widely recognized limitation of TMR, however, is the inherent size mismatch that occurs at the repair site between the larger caliber proximal nerve that is injured and the smaller caliber distal nerve. This size mismatch results in axonal escape and neuroma formation at the repair site.

### SUMMARY

In some aspects, the presently disclosed subject matter provides a nerve conduit comprising a tubular body having a proximal aperture and a distal aperture, wherein the proximal aperture has a diameter greater than a diameter of the distal aperture. In some aspects, the tubular body has a shape selected from a conical shape, a conical concave shape, and a conical convex shape. In certain aspects, the tubular body may be a right circular cone that has a right conical shape.

In some aspects, nerve conduit comprises a hydrogel. In certain aspects, the hydrogel further comprises one or more of a fibrin-, a collagen-, a tissue matrix-derived hydrogel, or combinations thereof. In particular aspects, the tubular body of the nerve conduit or hydrogel comprises one or more agents for inhibiting axonal growth, polarizing macrophages to the pro-regenerative phenotype, supporting angiogenesis, and combinations thereof. In more particular aspects, the tubular body or hydrogel comprises one or more components selected from a nanofiber hydrogel composite (NHC), and one or more bioactive agents that inhibit axonal outgrowth, such as semaphorin, a myelin-associated glycoprotein, and one or more chondroitin sulfate proteoglycans (CSPGs). In certain aspects, the hydrogel comprises an interpenetrating network (IPN).

In some aspects, the tubular body comprises a wall comprising a nanofiber diameter and pore size sufficient to allow diffusion of nutrients while preventing inflammatory macrophage infiltration. In certain aspects, the tubular body comprises a smooth wall, a crimped wall, or combinations thereof. In some aspects, the tubular body further comprises one or more extensions including a proximal extension, a distal extension, and combinations thereof. In certain aspects, the extension is adapted for suturing.

In other aspects, the presently disclosed subject matter provides a method for treating or repairing a nerve injury in a subject in need of treatment thereof, the method comprising: providing a presently disclosed nerve conduit; and contacting a large caliber injured nerve with the proximal aperture of the nerve conduit and contacting a small caliber sensory nerve with the distal aperture of the nerve conduit. In some aspects, the nerve comprises a peripheral nerve. In some aspects, the empty nerve conduit or hydrogel-filled nerve conduit can provide a mechanical guide for orderly, tapered axonal regeneration across a size-mismatched nerve coaptation site.

In some aspects, the nerve conduit comprises a conical nerve conduit comprising a CSPG-containing hydrogel and the method comprises targeted muscle reinnervation (TMR) or preventing neuroma in a subject with an injured nerve(s), such as following limb amputation.

In some aspects, the method comprises targeted sensory reinnervation (TSR) for painful neuroma prevention/treatment, wherein the large caliber injured nerve is coapted to the small caliber sensory nerve.

In some aspects, the method comprises targeted muscle reinnervation (TMR) for preventing neuroma, wherein the lumen of the nerve conduit comprises hydrogel only or does not include hydrogel.

In some aspects, the method comprises targeted sensory reinnervation (TSR) for afferent sensory input from a prosthesis.

In some aspects, the method comprises size-mismatched motor and sensory nerve transfers for motor and sensory functional restoration.

In some aspects, the method comprising targeted muscle reinnervation (TMR). In some aspects, the nerve conduit comprises an empty conduit or a hydrogel-filled conduit without CSPGs. In some aspects, the method maximizes the number of motor axons to innerve a target muscle. In some aspects, the method improves signal transduction from the target muscle. In some aspects, the target muscle is used for efferent signal amplification for prosthesis control.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1 shows a photograph of an example of a targeted muscle reinnervation (TMR) site that shows TMR of the median nerve (MN) to motor nerve to the short head of the biceps (MCN-SH) and further showing a significant size mismatch between the MN and motor nerve (from Gart et al., 2015);

FIG. 2 illustrates an isometric view of an embodiment of the presently disclosed biodegradable nanofiber conical conduit for nerve repair that tapers substantially linearly from the proximal aperture to the distal aperture;

FIG. 3 illustrates an isometric view of an embodiment of the presently disclosed biodegradable nanofiber conical conduit for nerve repair including a conical concave shape;

FIG. 4 illustrates an isometric view of an embodiment of the presently disclosed biodegradable nanofiber conical conduit for nerve repair including a conical convex shape;

FIG. 5 illustrates an isometric view of an embodiment of the presently disclosed biodegradable nanofiber conical conduit for nerve repair including proximal and distal extensions;

FIG. 6 illustrates an isometric view of an embodiment of the presently disclosed biodegradable nanofiber conical conduit for nerve repair including an arrangement of lateral ridges for crimping action;

FIG. 7 illustrates an isometric view of an embodiment of the presently disclosed biodegradable nanofiber conical conduit for nerve repair including an arrangement of radial ridges for crimping action;

FIG. 8 illustrates an embodiment of a presently disclosed conical shaped nerve conduit comprising a crimped wall;

FIG. 9 illustrates an isometric view of an embodiment of the presently disclosed biodegradable nanofiber conical conduit for nerve repair filled with hydrogel for inhibiting excessive axonal growth;

FIG. 10 and FIG. 11 are various views showing more details of the presently disclosed biodegradable nanofiber conical conduits for nerve repair;

FIG. 12A shows a photograph of an example of the presently disclosed conical conduit having a 1.5-mm proximal diameter, 0.5-mm distal diameter, and 10-mm length;

FIG. 12B shows a photograph of an example of a positive conical conduit mold with 1.5-mm proximal diameter, 0.5-mm distal diameter, and 15-mm length taper;

FIG. 13A shows a photograph of an example of the presently disclosed conical conduit having a 1.5-mm proximal diameter, 0.5-mm distal diameter, and 10-mm length connecting a severed rat sciatic nerve stump and tibial nerve target;

FIG. 13B shows a photograph of an example of the size mismatch of a severed sciatic nerve and tibial branch;

FIG. 13C shows a photograph of an example of the implementation of the presently disclosed conical conduit that is micro-sutured to the sciatic nerve and the tibial nerve;

FIG. 14A shows a photograph of an example of a proximal sciatic nerve stump coapted to distal tibial nerve fascicle supplying the lateral gastrocnemius muscle, modeling the coaptation size-mismatch that occurs in TMR clinically;

FIG. 14B shows a photograph of an example of a neuroma formation at the surgical site 12-weeks post-TMR;

FIG. 14C shows a photograph of an example of a CSPG-conduit implanted within a size-mismatched coaptation site;

FIG. 14D shows a photograph of an example of a tapered reinnervation without gross evidence of the neuroma formation at the surgical site 12-weeks post TMR with implanted CSPG-conduit;

FIG. 15 shows a photograph of an example of how TUJ1-staining of longitudinal nerve sections demonstrates orderly, tapered axonal growth without axonal escape and neuroma formation in the CSPG-conduit group;

FIG. 16 shows the greater mean gastrocnemius mass indicates greater functional nerve recovery obtained in the CSPG-conduit group compared with the negative control (neuroma group); and

FIG. 17 demonstrates eliciting Tinel's sign to assess pain at the coaptation site. Statistical analysis was performed using an ordinary one-way ANOVA with Tukey's post hoc test. Bars represent mean±SEM (ns=not significant, **p<0.01, ****p<0.0001). At week 23, sample sizes (n) are 6, 7, 9, 10, and 4 for Neuroma, Direct Repair, Conduit, CSPG-loaded Conduit, and Sham Surgery Control groups, respectively.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

### I. Biodegradable Nanofiber Conical Conduits for Nerve Repair and Methods of Using Same

In some embodiments, the presently disclosed subject matter provides biodegradable nanofiber conical conduits for nerve repair and methods of using same.

In some embodiments, the presently disclosed biodegradable nanofiber conical conduits for nerve repair and methods provide a suturable conduit having a conical shape/geometry including a larger proximal aperture and smaller distal aperture to mechanically guide the regenerating axons across the mismatched repair and thereby prevent axonal escape and neuroma formation. By contrast, current conduits known in the art are not tapered (i.e., proximal and distal ends have substantially the same diameters) and are meant only to bridge the gap of an injured nerve.

In some embodiments, the presently disclosed biodegradable nanofiber conical conduits for nerve repair and methods provide a tubular body or hydrogel lumen that can inhibit excessive axonal growth from the large proximal nerve stump that might otherwise overwhelm the synaptic capacity of the smaller distal nerve stump.

Accordingly, in some embodiments, the presently disclosed biodegradable nanofiber conical conduits for nerve repair and methods provide a nerve conduit comprising a tubular body having a proximal aperture and a distal aperture, wherein the proximal aperture has a diameter greater than a diameter of the distal aperture. In some embodiments, the tubular body has a shape selected from a conical shape, a conical concave shape, and a conical convex shape. In certain embodiments, the tubular body may be a right circular cone that has a right conical shape. Circular cone means that the base is circular. Right cone means that the axis passes through the center of the base at right angles to its plane. Contrasted with right cones are oblique cones. In particular embodiments, the right circular cone has a linear increase in diameter size from the distal aperture to the proximal aperture. In some embodiments, the tubular body has a conical shape that tapers in decreasing diameter from the proximal aperture to the distal aperture. In particular embodiments, the tubular body has a conical shape having a taper range of, for example, from about 1° to about 89°.

In some embodiments, the presently disclosed biodegradable nanofiber conical conduits for nerve repair and methods provide a lumen comprising a hydrogel. The term “lumen” is used interchangeably with the term “channel,” “inner space,” “cavity,” and the like to describe the inner volume of the conical conduit that can be filled with a hydrogel.

In particular embodiments, the tubular body or hydrogel comprises one or more agents for inhibiting axonal growth, polarizing macrophages to the pro-regenerative phenotype, supporting angiogenesis, and combinations thereof. In more particular embodiments, the tubular body or hydrogel comprises one or more components selected from a nanofiber hydrogel composite (NHC), and one or more bioactive agents that inhibit axonal outgrowth, such as semaphorin, a myelin-associated glycoprotein, and one or more chondroitin sulfate proteoglycans (CSPGs).

In certain embodiments, the NHC comprises functionalized poly(ε-caprolactone) (PCL) fiber fragments distributed in and covalently conjugated to a hydrogel network formed by reacting acrylated hyaluronic acid (HA) with thiolated poly(ethylene glycol) (PEG-SH). In certain embodiments, the one or more agents is selected from acrylated hyaluronic acid (HA-Ac) crosslinked with thiolated poly(ethylene glycol) (PEG-SH), HA-Ac crosslinked with PEG-SH and one or more chondroitin sulfate proteoglycans (CSPGs), and HA-Ac cross-linked with PEG-SH, CSPGs, and poly(ε-caprolactone) (PCL) nanofiber fragments. In particular embodiments, the hydrogel has an overall stiffness (storage modulus, G′) ranging from about 50 Pa to about 500 Pa.

In certain embodiments, the hydrogel comprises an interpenetrating network (IPN). In certain embodiments, the hydrogel further comprises one or more of a fibrin-, a collagen-, a tissue matrix-derived hydrogel, or combinations thereof.

In some embodiments, the tubular body comprises a wall comprising a nanofiber diameter and pore size sufficient to allow diffusion of nutrients while preventing inflammatory macrophage infiltration. In certain embodiments, the tubular body comprises a nanofiber mesh wall having a substantially uniform thickness ranging from about 50 μm to about 500 μm and with a pore size of less than about 10 μm. In certain embodiments, the nanofiber mesh wall comprises randomly oriented nanofibers having a diameter ranging from about 100 nm to about 2 μm.

In some embodiments, the nanofiber mesh wall comprises a synthetic material selected from poly(ε-caprolactone) (PCL), copolymers of ε-caprolactam and hexamethylendiaminadipate, polyglycolic acid (PGA), poly(lactic acid) (PLA), poly (1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), and polyhydroxyoctanoate (PHO), as well as co-polymers, blends, analogs, derivatives, modifications, and mixtures thereof.

In some embodiments, the nanofiber mesh wall comprises a natural material selected from hyaluronic acid (HA), silk, keratin, collagen, gelatin, fibrinogen, elastin, actin, myosin, cellulose, amylose, dextran, chitin, glycosaminoglycans (GAG), deoxyribonucleic acids (DNA), ribonucleic acids (RNA), chitin, chitosan (CS), alginate, as well as co-polymers, blends, analogs, derivatives, modifications, and mixtures thereof.

In some embodiments, the tubular body comprises a smooth wall, a crimped wall, or combinations thereof. In certain embodiments, the crimped wall comprises one or more ridges characterized by a kink-resistance of up to a 90° bend and a length adjustability of less than or equal to 100% of an initial length of the conduit.

In some embodiments, the tubular body further comprises one or more extensions including a proximal extension, a distal extension, and combinations thereof. In certain embodiments, the extension is adapted for suturing. In particular embodiments, the extension has one or more dimensions ranging in diameter from about 50 μm to about 25 mm and in length from about 0 mm to about 50 mm.

In some embodiments, the proximal aperture and the distal aperture have a diameter ranging from about 50 μm to about 25 mm, provided that the diameter of the proximal aperture is greater than the diameter of the distal aperture. In certain embodiments, the proximal aperture has a diameter of about 1.5 mm and the distal aperture has a diameter of about 0.5 mm. In certain embodiments, the tubular body has a length ranging from about 5 mm to about 50 mm. In particular embodiments, the tubular body has a length of about 10 mm. In more particular embodiments, the tubular body has one or more dimensions comprising a 1.5-mm proximal aperture diameter, a 0.5-mm distal aperture diameter, and a 10-mm length.

In other embodiments, the presently disclosed subject matter provides a method for treating or repairing a nerve injury in a subject in need of treatment thereof, the method comprising: providing a presently disclosed nerve conduit; and contacting a large caliber injured nerve with the proximal aperture of the nerve conduit and contacting a small caliber sensory nerve with the distal aperture of the nerve conduit. In some embodiments, the nerve comprises a peripheral nerve. In some embodiments, the empty nerve conduit or hydrogel-filled nerve conduit can provide a mechanical guide for orderly, tapered axonal regeneration across a size-mismatched nerve coaptation site.

As used herein, the term “treating” including mitigating, minimizing, or preventing formation of neuromas in severed, including divided or sectioned, or damaged nerve endings, in particular, peripheral nerve endings, including managing pain associated with severed or damaged nerve endings.

The terms “minimizing” or “mitigating” are intended to mean that the use of the presently disclosed nerve conduit on a nerve ending substantially reduces pain and severity of any symptoms associated with neuromas, i.e., clusters of disorganized nerve fibers, while not necessarily completely preventing or inhibiting formation of a neuroma over time. While some disorganized neural growth may still occur over time, the use of a presently disclosed nerve conduit in accordance with certain aspects of the present disclosure advantageously reduces symptoms and pain as compared to conventional neuroma treatment techniques known in the art.

In some embodiments, the nerve conduit comprises a conical nerve conduit comprising a CSPG-containing hydrogel and the method comprises targeted muscle reinnervation (TMR) for preventing neuroma in a subject with an injured nerve(s). In some embodiments, the injured nerve(s) is a result of limb amputation.

In some embodiments, the method comprises targeted sensory reinnervation (TSR) for painful neuroma prevention/treatment, wherein the large caliber injured nerve is coapted to the small caliber sensory nerve.

In some embodiments, comprises targeted muscle reinnervation (TMR) for preventing neuroma, wherein the lumen of the nerve conduit comprises hydrogel only or does not include hydrogel.

In some embodiments, the method comprises targeted sensory reinnervation (TSR) for afferent sensory input from a prosthesis.

In some embodiments, the method comprises size-mismatched motor and sensory nerve transfers for motor and sensory functional restoration. In some embodiments, an empty nerve conduit or hydrogel-filled conduit can be used in facilitating repair of size-mismatched motor and sensory nerve transfers for motor and sensory functional restoration.

In some embodiments, the method comprising targeted muscle reinnervation (TMR). In some embodiments, the nerve conduit comprises an empty conduit or a hydrogel-filled conduit without CSPGs. In some embodiments, the method maximizes the number of motor axons to innerve a target muscle. In some embodiments, the method improves signal transduction from the target muscle. In some embodiments, the target muscle is used for efferent signal amplification for prosthesis control.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Biodegradable Nanofiber Conical Conduits for Targeted Muscle Reinnervation

1.1 Overview. Nerve injury occurs commonly through a variety of mechanisms, including trauma and surgical injury. When a peripheral nerve is injured, axons within the proximal nerve stump will begin regenerating in an attempt to reinnervate distal muscle and skin and thereby restore motor and sensory function. When the injured nerve is not immediately repaired, however, as is often the case, the axons regenerating from the proximal nerve stump will not find a distal nerve to grow into and will instead form an aggregate of disorganized neural growth called a neuromas. Cravioto and Battista, 1981; Weng et al., 2016; Lu et al., 2018; Dellon and Mackinnon, 1986; Wood, 1829; Morton's neuroma surgery, 2018. Many of these neuromas result in debilitating pain that is very difficult to treat.

Among the many causes of nerve injury resulting in symptomatic neuroma formation, extremity amputation is particularly problematic, given that multiple large caliber nerves are necessarily transected and left in discontinuity. As a result, amputees tend to suffer from severe, intractable neuroma pain, with estimates of prevalence ranging from 25% to 75%. Cravioto and Battista, 1981; Weng et al., 2016; Bowen et al., 2017.

Extremity amputations are common operations both in the United States and in other areas of the world. It is estimated that there are nearly 2 million adult amputees living in the United States alone. The underlying causes of extremity amputation are diverse, the most common being complications of type 2 diabetes, non-diabetic peripheral vascular disease, trauma, and oncologic conditions. Cravioto and Battista, 1981; Weng et al., 2016; Lu et al., 2018. Given the increasing prevalence of several of these diseases, conservative estimates suggest that the population of people living with amputated limbs will likely double within the next several decades. Dellon and Mackinnon, 1986.

One of the most promising treatments to emerge for the treatment of neuromas is ‘targeted muscle reinnervation’ (TMR), a procedure that involves suturing the injured proximal nerve stump to the distal nerve stump of a freshly-cut, small-caliber motor nerve as it enters muscle. Dumanian et al., 2019. This procedure allows the axons regenerating from the injured proximal nerve stump that would otherwise form a neuroma to instead regenerate through the distal motor nerve into muscle. This approach has rapidly gained in popularity and has become the preferred surgical approach to prevent and treat symptomatic neuromas. A recently published randomized, controlled trial demonstrated the strong treatment effect of TMR, so much so that the trial concluded prematurely due to the superiority of the TMR compared to the control group. Dumanian et al., 2019.

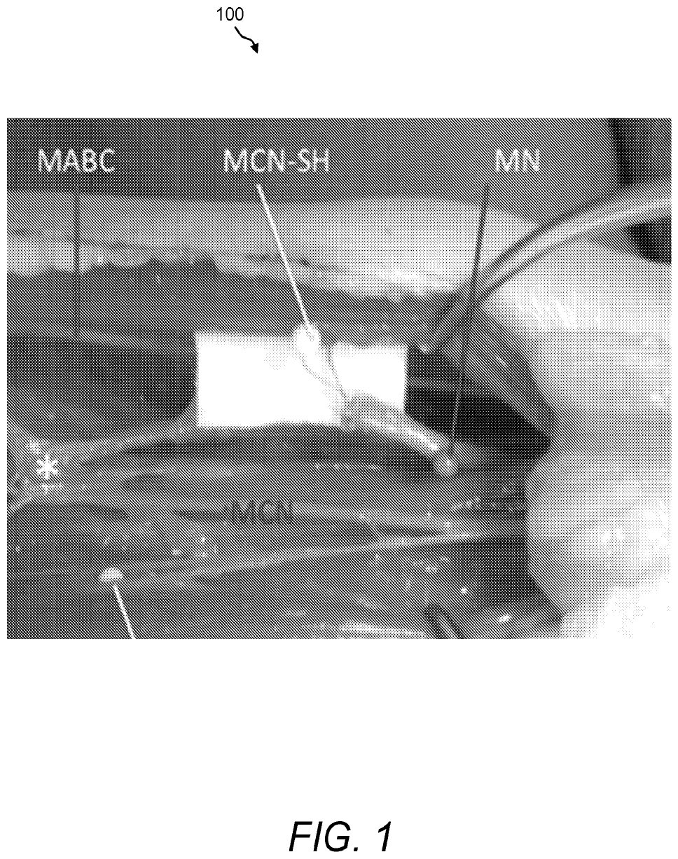

Despite these promising results, the same report showed that roughly 30% of patients still struggled with chronic pain following TMR. This observation is likely due to a major limitation inherent to TMR. When performing TMR, there is a very large size mismatch between the large-caliber proximal nerve stump and the small motor nerve to which it is sutured, often on a scale of 5-to-1 or more, as shown, for example, in FIG. 1. As a consequence of this size mismatch, many of the axons generating from the large proximal nerve stump fail to regenerate into the small distal nerve and these escaped axons will form a neuroma at the repair site. Bergmeister et al., 2019; Mackinnon, 1989. Thus, there is a need to devise a solution to prevent axons from escaping from the size-mismatched repair site and forming a painful neuroma.

Designs known in the art for addressing peripheral nerve injury and neuroma have primarily focused on preventing neuroma formation through nerve caps with autologous tissue grafts or regenerating peripheral nerves through straight nerve conduits. Processes of manufacture for peripheral nerve guides have included 3D biomimetic nerve conduits, nanofiber conduits made from polymer-protein mixtures, immunosuppression for nerve allografts, and polymer-magnesium conduits. See for example, U.S. Pat. No. 10,405,963 for Method of producing a 3D subject specific biomimetic nerve conduit, to McAlpine et al., issued Sep. 10, 2019; U.S. Pat. No. 8,728,817 for Compositions and methods for making and using laminin nanofibers, to Ogle et al., issued May 20, 2014; U.S. Patent Application Publication No. 2020/0030237A1, for Localized Jan. 30, 2020; U.S. Pat. No. 9,925,695, for Poly(lactic-co-glycolic acid (PLGA) composites with magnesium wires enhanced networking of primary neurons, to Liu et al., issued Mar. 27, 2018. Device designs specifically addressing neuroma formation have utilized nerve caps for preventing neuroma formation. U.S. Patent Application Publication No. 2020/0030237A1, for Localized immunosuppression of allografts for peripheral nerve repair, to Bushman, published Jan. 30, 2020

Previous research also has investigated the use of silicone tubes to prevent inflammatory cell infiltration after peripheral nerve injury. Okuda et al., 2006. Although each of these devices and solutions address peripheral nerve injury and neuroma prevention, none have gained traction as it is not possible to completely block neuroma formation with a cap. Furthermore, none of the previous designs specifically focus on targeted muscle reinnervation and the size-mismatch issue between the proximal and distal nerve stumps.

1.2 Design Features and Function

Referring now to FIG. 1, is a photograph of an example of a TMR site 100 that shows TMR of the median nerve (MN) to motor nerve to the short head of the biceps (MCN-SH) and further showing a significant size mismatch between the MN and motor nerve.

TMR site 100 shown in FIG. 1 is an example of a TMR procedure site that may benefit from the presently disclosed biodegradable nanofiber conical conduits for TMR and methods. For example, the presently disclosed biodegradable nanofiber conical conduits for TMR and methods may include, but are not limited to, the following design features: (1) a suturable conduit having a conical shape/geometry including a larger proximal aperture and smaller distal aperture to mechanically guide the regenerating axons across the mismatched repair and thereby prevent axonal escape and neuroma formation; (2) a hydrogel lumen, or the tubular body itself, that can adequately inhibit excessive axonal growth from the large proximal nerve stump that might otherwise overwhelm the capacity of the smaller distal nerve stump; and (3) immunomodulatory properties that downregulate the inflammatory response via polarization of macrophages from an inflammatory to a pro-regenerative phenotype.

Collectively these features prevent axonal escape and painful neuroma formation at the TMR repair site and thereby improve the treatment response and success rate of this already widely-adopted surgical approach. Importantly, surgeons performing TMR, who are already well-versed in the use of non-conical conduits for other types of nerve repair, are likely to find that the presently disclosed biodegradable nanofiber conical-shaped conduits facilitate the technical execution of performing TMR repair.

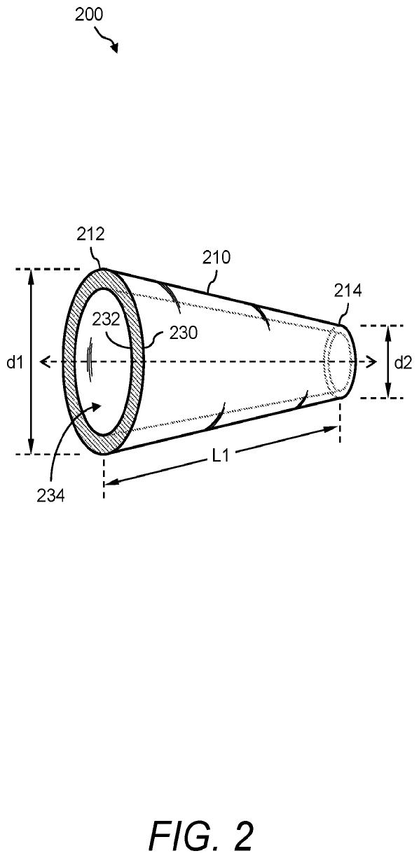

Referring now to FIG. 2 through FIG. 11 are isometric views of representative embodiments of a conical conduit 200, which are examples of the presently disclosed biodegradable nanofiber conical conduits for nerve repair. For example, FIG. 2 shows an embodiment of conical conduit 200 for nerve repair that tapers substantially linearly. Conical conduit 200 includes tubular body 210 having a proximal aperture 212 and a distal aperture 214. Tubular body 210 of conical conduit 200 has a length L. In one embodiment, the length L of tubular body 210 may be from about 5 mm to about 50 mm. In another embodiment, the length L of tubular body 210 may be about 10 mm.

Tubular body 210 of conical conduit 200 can include an outer wall 230, and inner wall 232, which define a hollow channel, or lumen, 234, which can be filled, for example, with a hydrogel.

In one embodiment, outer wall 230 of tubular body 210 may include a nanofiber diameter and pore size sufficient to allow diffusion of nutrients while preventing inflammatory macrophage infiltration. For example, outer wall 230 of tubular body 210 may comprise a nanofiber mesh wall having a substantially uniform thickness ranging from about 50 μm to about 500 μm and with a pore size of less than about 10 μm. In certain embodiments, the nanofiber mesh wall comprises randomly oriented nanofibers having a diameter ranging from about 100 nm to about 2 μm.

Proximal aperture 212 has a diameter d1 and distal aperture 214 has a diameter d2, wherein diameter d1 of proximal aperture 212 is greater than diameter d2 of distal aperture 214. Further, diameter d1 of proximal aperture 212 and diameter d2 of distal aperture 214 may range from about 50 μm to about 25 mm, provided that diameter d1 of proximal aperture 212 is greater than diameter d2 of distal aperture 214. In one embodiment, proximal aperture 212 has a diameter d1 of about 1.5 mm and distal aperture 214 has a diameter d2 of about 0.5 mm. In a specific embodiment, conical conduit 200 has one or more dimensions including a 1.5-mm proximal aperture diameter, a 0.5-mm distal aperture diameter, and a 10-mm length.

Further, conical conduit 200 shown in FIG. 2 is an embodiment of a right circular cone that has a right conical shape. Circular cone means that the base is circular. Namely, conical conduit 200 is a right circular cone that has a linear increase in diameter size from distal aperture 214 to proximal aperture 212. Tubular body 210 of conical conduit 200 has a conical shape that tapers from proximal aperture 212 to distal aperture 214. For example, conical conduit 200 has a conical shape having a taper range of from about 1° to about 89°.

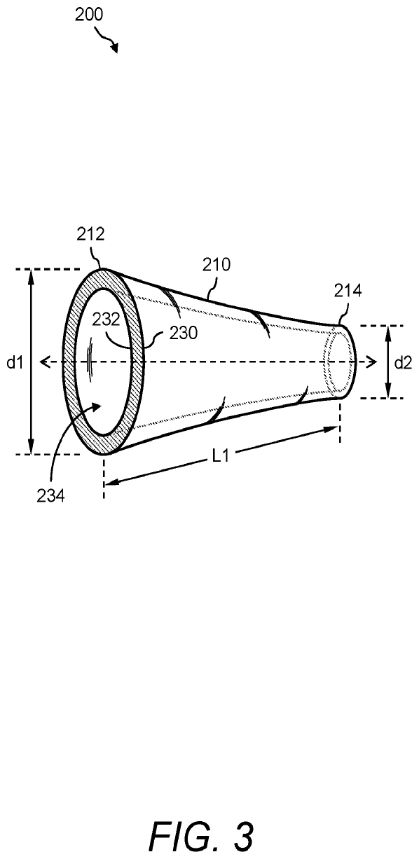

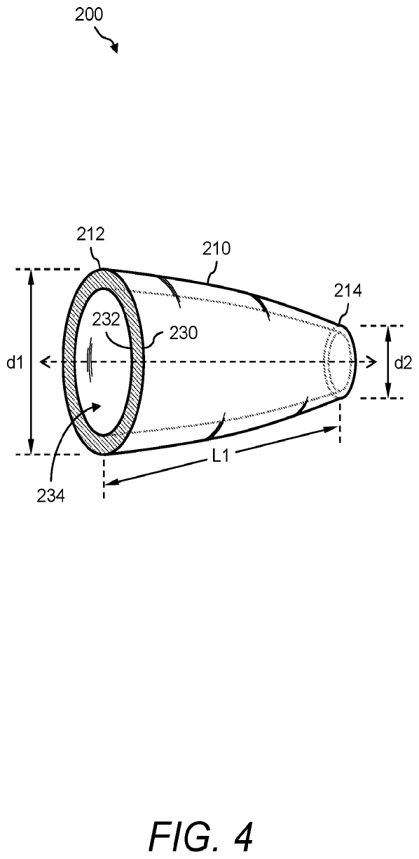

While the conical conduit 200 shown in FIG. 2 is a right circular cone with a linear increase in diameter size, the structure of the conduit may be modified to include a concave or convex characteristic to adjust the rate at which the nerve axons taper during the regenerative process. For example, FIG. 3 shows an embodiment of a conical conduit 200 having a conical concave shape. Further, FIG. 4 shows an embodiment of a conical conduit 200 having a conical convex shape.

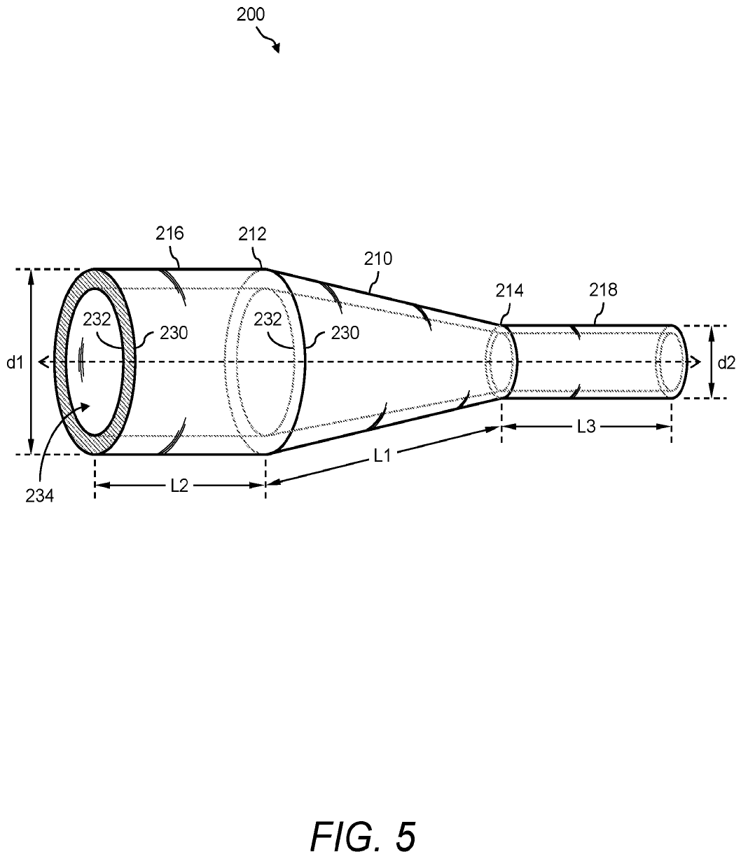

In yet another embodiment and referring now to FIG. 5, conical conduit 200 may include proximal and distal extensions. For example, conical conduit 200 may include a proximal extension 216 extending from proximal aperture 212 of tubular body 210. Further, conical conduit 200 may include a distal extension 218 extending from distal aperture 214 of tubular body 210. Additionally, both proximal aperture 212 and proximal extension 216 may have the diameter d1 and both distal aperture 214 and distal extension 218 may have the diameter d2. Proximal extension 216 and distal extension 218 may be adapted for suturing. For example, proximal extension 216 and distal extension 218 have one or more dimensions ranging in diameter from about 50 μm to about 25 mm and in length from about 0 mm to about 50 mm.

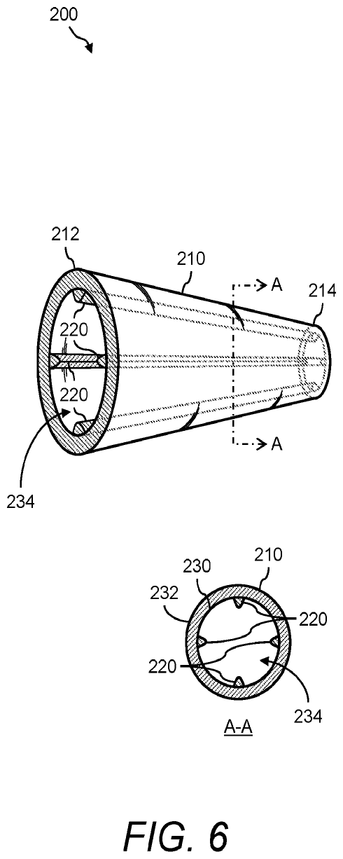

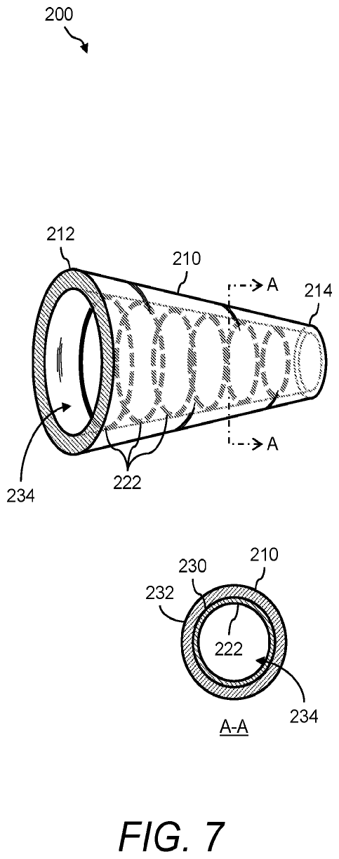

In yet another embodiment and referring now to FIG. 6 and FIG. 7, conical conduit 200 may include an arrangement of crimping features. For example, FIG. 6 shows an arrangement of lateral ridges 220 running along and protruding from the inner wall 232 of tubular body 210 of conical conduit 200 and wherein lateral ridges 220 are provided for crimping action. Additionally, FIG. 7 shows an arrangement of radial ridges 222 arranged around and protruding from inner wall 232 of tubular body 210 of conical conduit 200 and wherein radial ridges 222 are provided for crimping action. Conical conduit 200 may include any number and spacing of lateral ridges 220 or radial ridges 222 and wherein lateral ridges 220 and radial ridges 222 may be any desired width, height, and shape.

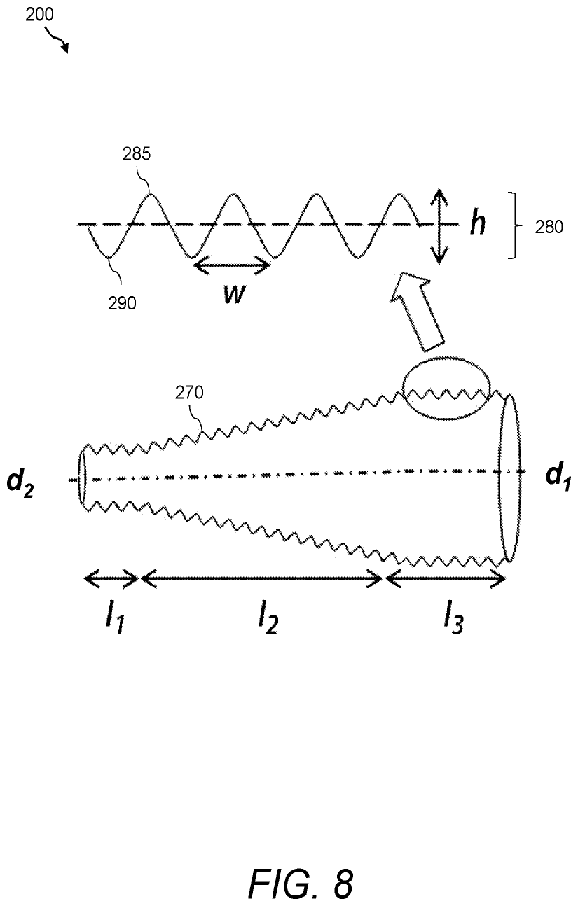

Referring now to FIG. 8, in some embodiments, the presently disclosed nerve conduit comprises a crimped wall 270. Crimped wall 270 can comprise a crimp pattern 280 having a plurality of crests 285 and troughs 290. In some embodiments, the plurality of crests 285 and troughs 290 of crimp pattern 280 of crimped wall 270 has a thickness (h), a width (w), a first diameter (d₁), i.e., a diameter at the proximal end of the nerve conduit, a second diameter (d₂), i.e., a diameter at the distal end of the nerve conduit, a first length (l₁), a second length (l₂), and a third length (l₃). Thickness (h) corresponds to the height measured from crest 285 to trough 290 and also corresponds to the thickness of crimped wall 270. Width (w) corresponds to the distance between the maximum of one crest 285 to the maximum of another crest 285 or the minimum of one trough 290 to the minimum of another trough 290.

In some embodiments, h has a range of about 0.1 mm to 5.0 mm, including about 0.1, 0.2, 0.3, 0.4, 0.5, 0.6, 0.7, 0.8, 0.9, 1.0, 1.5, 2.0, 2.5, 3.0, 3.5, 4.0, 4.5, and 5.0 mm, with a ratio of w to h having a range of about 0.1 to about 10, including about 0.1, 0.2, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0; d₁ has a range of about 0.5 mm to about 25 mm, including about 0.5, 1, 2, 3, 4, 5, 6, 7, 8, 9, 10, 11, 12, 13, 14, 15, 16, 17, 18, 19, 20, 21, 22, 23, 24, and 25 mm, d₂ has a range of about 0.05 mm to about 10 mm, including about 0.1, 0.2, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 mm, and wherein d₂<d₁; and l₁ has a range of about 0 mm to about 10 mm, including about 0.0, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 mm, l₂ has a range of about 2 mm to about 50 mm, including about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mm, and l₃ has a range of about 0 mm to about 10 mm, including about 0.0, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 mm.

In some embodiments, thickness (h) is about 0.5 mm to about 2 mm, including about 0.5, 0.6, 0.7, 0.8, 0.9, 1.0, 1.1, 1.2, 1.3, 1.4, 1.5, 1.6, 1.7, 1.8, 1.9, and 2.0 mm; width (w) is about 0.5 mm to about 1 mm, including about 0.5, 0.6, 0.7, 0.8, 0.9, 1.0 mm; first diameter (d₁) is about 5 mm to about 10 mm, including about 5.0, 5.5, 6.0, 6.5, 7.0, 7.5, 8.0, 8.5, 9.0, 9.5, and 10.0 mm; second diameter (d₂) is about 1 mm to about 4 mm, including about 1.0, 1.5, 2.0, 2.5, 3.0, 3.5, and 4.0 mm; first length (l₁) is about 2 mm to about 5 mm, including about 2.0, 2.5, 3.0, 3.5, 4.0, 4.5, and 5.0 mm; second length (l₂) is about 5 mm to about 20 mm, including about 5, 6, 7, 8, 9, 10, 11, 12, 13, 14, 15, 16, 17, 18, 19, and 20 mm; and third thickness (l₃) is about 2 mm to about 5 mm, including about 2.0, 2.5, 3.0, 3.5, 4.0, 4.5, and 5.0 mm.

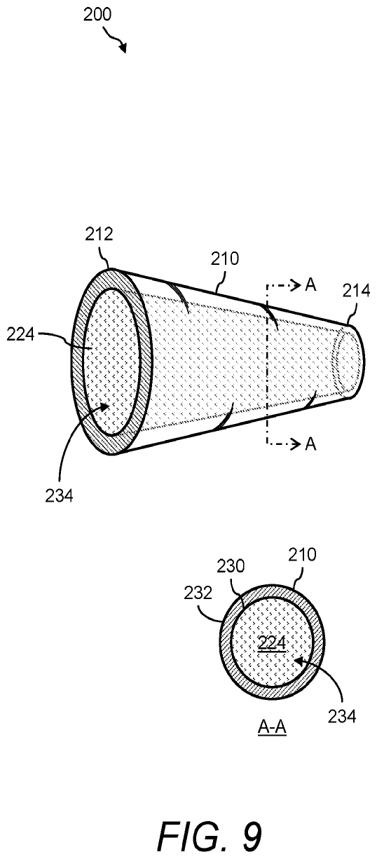

In yet another embodiment and referring now to FIG. 9, channel 234 of conical conduit 200 may filled with hydrogel for inhibiting excessive axonal growth. For example, conical conduit 200 may filled with a quantity of hydrogel 224 that substantially fills the space inside tubular body 210, i.e., channel 234. Hydrogel 224 may be provided to adequately inhibit excessive axonal growth from the large proximal nerve stump that might otherwise overwhelm the synaptic capacity of the smaller distal nerve stump. Tubular body 210 also can inhibit excessive axonal growth.***

In one embodiment, hydrogel 224 includes an interpenetrating network (IPN). For example, the tubular body, the hydrogel, and/or the IPN may include one or more agents for inhibiting axonal growth, polarizing macrophages to the pro-regenerative phenotype, supporting angiogenesis, and combinations thereof. More specifically, the IPN may include an NHC and one or more CSPGs. Further, hydrogel 224 may have an overall stiffness (storage modulus, G′) ranging from about 50 Pa to about 500 Pa.

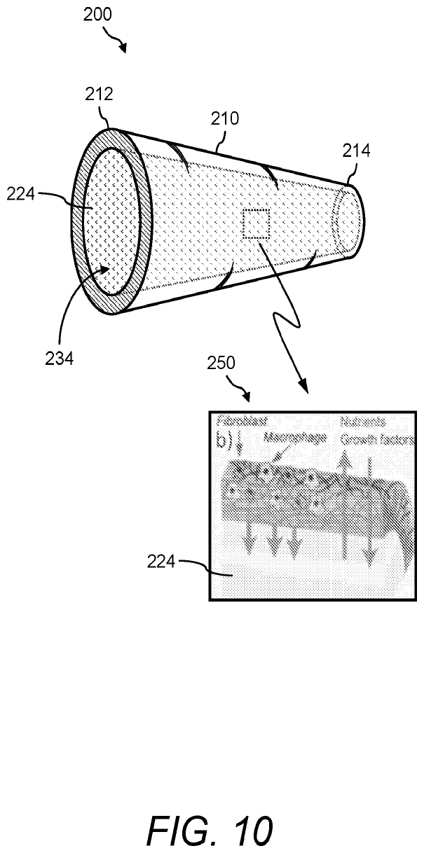

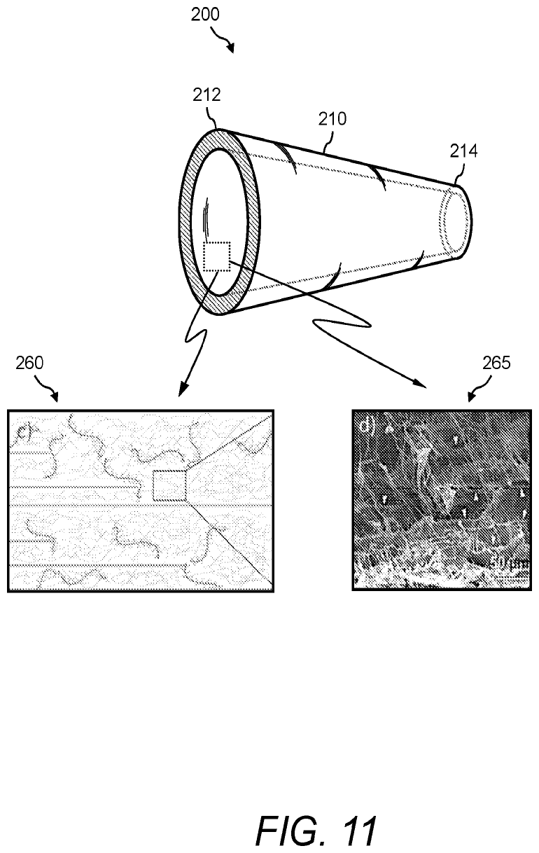

Referring now to FIG. 10 and FIG. 11 is various views showing more details of the presently disclosed biodegradable nanofiber conical conduits for nerve repair. For example, FIG. 10 shows a magnified view of a portion of conical conduit 200. For example, a panel 250 shows the conduit wall with sufficient nanofiber diameter and pore size to allow diffusion of nutrients while preventing inflammatory macrophage infiltration. FIG. 11 shows magnified views of a portion of conical conduit 200. For example, a panel 260 shows IPN gel with physically entangled NHC and CSPGs, in which the CSPGs inhibit axonal growth. Further, a panel 265 shows that NHC mimics ECM structure.

1.2.1 Conical Conduit

A suturable, conical-shaped nerve conduit addresses the repair site size-mismatch limiting the efficacy of nerve repair. It does so by mechanically guiding regenerating axons across the repair site and preventing them from escaping the repair site and forming a painful neuroma.

The presently disclosed conduit is characterized by its conical shape, which is defined by a tubular body having a larger opening, or aperture, and a smaller opening, or aperture, of contrasting diameters, each ranging between about 50 μm and about 25 mm. The conduit opening diameters encompass the range of diameters for nerves that may be inserted into either opening of the conduit for nerve repair.

In one embodiment, the conduit proximal diameter is about 1.5 mm and the conduit distal diameter is about 0.5 mm. The length of the conical conduit ranges between about 5 mm to about 50 mm, with a taper range of between about 1° to about 89°. In one embodiment, the length of the cone is about 10 mm. Though the current conduit structure is a right circular cone with a linear increase in diameter size, the structure of the conduit may be modified to include a concave or convex characteristic to adjust the rate at which the nerve axons taper during the regenerative process. The variation in diameter of the openings allow for the taper of the conduit, which in turn tapers the regenerating axons, thereby addressing the size-mismatch of the peripheral nerve stump and the distal nerve target.

The presently disclosed conical conduits may be elongated with tubular extensions for increased surface area, allowing for easier suturing. The extensions may be immediately attached to either the larger or smaller opening, or both, ranging in diameter from about 50 μm and to about 25 mm and in length from about 0 mm to about mm. In one embodiment, the extensions are 0 mm in length.

In some embodiments, the presently disclosed conical nerve conduit includes a nanofiber mesh wall with a relatively uniform thickness ranging from about 50 μm to about 500 μm and with a pore size of less than about 10 μm. In some embodiments, the presently disclosed conical conduit includes randomly oriented nanofibers having a fiber diameter ranging from about 100 nm to about 2 μm in diameter. Previous findings suggest that these ranges of fiber diameters and pore sizes are ideal for entrapping and promoting macrophage polarization towards the M2 pro-regenerative phenotype, thereby reducing the inflammatory reaction towards the conduit. Sarhane et al., 2019. Furthermore, the porosity of the conduit allows for adequate nutrient diffusion through the conduit wall.

The wall of the conduit can be either smooth or crimped in nature. The matrix of the conduit can be disposed to form ridges (crimping) characterized by both kink-resistance of up to a 90° bend and length adjustability of less than or equal to 100% of the initial conduit length. This feature allows the conduit to tailor to multiple cases of nerve size mismatch.

The nanofiber matrix may include a range of acceptable synthetic and natural polymers for medical applications, including their composites. Preferred synthetic materials for the matrix include, but are not limited to, poly(ε-caprolactone) (PCL), copolymers of ε-caprolactam and hexamethylendiaminadipate, polyglycolic acid (PGA), poly(lactic acid) (PLA), poly (1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), and polyhydroxyoctanoate (PHO), as well as co-polymers, blends, analogs, derivatives, modifications, and mixtures thereof. Al-Enizi et al., 2018; U.S. Pat. No. 8,728,817 for Compositions and methods for making and using laminin nanofibers, to Ogle et al., issued May 20, 2014.

Other representative natural materials for the matrix include, but are not limited to, hyaluronic acid (HA), silk, keratin, collagen, gelatin, fibrinogen, elastin, actin, myosin, cellulose, amylose, dextran, chitin, glycosaminoglycans (GAG), deoxyribonucleic acids (DNA), ribonucleic acids (RNA), chitin, chitosan (CS), alginate, as well as co-polymers, blends, analogs, derivatives, modifications, and mixtures thereof. Al-Enizi et al., 2018; Painter and Coleman, 2008.

1.2.2 Conduit Filler

The gel filling of the conduit, or the tubular body itself, aims to inhibit axonal growth, polarize macrophages to the pro-regenerative phenotype, and support angiogenesis, or any combination of these functions. Representative formulations for the gel filling include acrylated hyaluronic acid (HA-Ac) crosslinked with thiolated poly(ethylene glycol) (PEG-SH), HA-Ac crosslinked with PEG-SH and chondroitin sulfate proteoglycans (CSPGs), and HA-Ac cross-linked with PEG-SH, CSPGs, and PCL nanofiber fragments. The gel has an overall stiffness (storage modulus, G′) ranging from Pa to 500 Pa. Li et al., 2020.

HA-Ac acts as a structural hydrogel that mimics native extracellular matrix (ECM), resembling the environment of the peripheral nerve. CSPGs have been shown to inhibit axonal growth for preventing neuroma formation. Lemons et al., 1999; Snow et al., 1990. Crosslinking of the PCL nanofiber fragments with hyaluronic acid has been shown to create a nanofiber-hydrogel composite (NHC) that promotes macrophages polarization to the pro-regeneration phenotype and angiogenesis. Sarhane et al., 2019.

The gel filling includes, but is not limited to the above materials and any other co-polymers, blends, analogs, derivatives, modifications, and mixtures thereof. Substitutes for CSPGs can include other molecules that similarly inhibit axonal growth and prevent neuroma formation. The NHC can be synthesized with hydrogels other than hyaluronic acid that resemble native ECM, such as collagen and fibrin. PEG-SH can be substituted by other chemical cross-linkers.

1.3 Process of Manufacture

1.3.1 Electrospinning of Nanofiber Scaffold

The current manufacturing process involves electrospinning a poly(ε-caprolactone) (PCL) polymer solution. In one implementation, an 8 wt % PCL solution using 80,000 molecular weight (MW) PCL in a 9:1 solvent of dichloromethane (DCM) and dimethylformamide (DMF), respectively is electrospun at 10 kV at a 10 cm distance from a mandrel rotating at 140 RPM. After electrospinning, the resulting fiber tube is sprayed with ethanol while on the mandrel to remove the fiber tube from the mandrel surface.

Manufacturing nanofibers of similar dimensions and properties, however, can be accomplished through methods including, but not limited to, rotary jet spinning of polymer solutions, phase separation of polymer solutions, polymer nanofiber self-assembly, magnetospinning polymer solutions, and melt blowing a polymer melt. Additionally, the polymer solution can range from 5 wt % to 20 wt % with PCL MW ranging from 15,000 to 100,000. Alternatives to PCL are listed above, and organic solvent alternatives can include other solvents capable of dissolving the polymer of choice, including but not limited to ethyl ether, hexane, tetrachloroethane, toluene, xylene, hexafluoro-2-propanol, and analogs, derivatives, modifications, and mixtures thereof. Electrospinning voltages can range from 5 kV to 24 kV, with spin distances ranging from 3 cm to 20 cm and mandrel rotation speeds ranging from 50 RPM to 1000 RPM.

1.3.2 Formation of Conical Shape

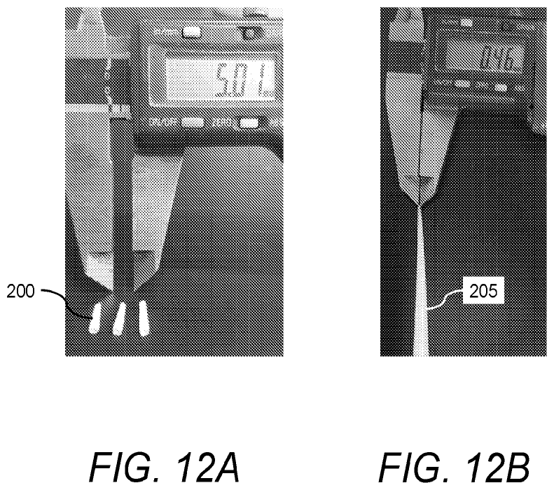

In one embodiment, the presently disclosed conical conduit is formed by cutting a PCL nanofiber scaffold tube into 1.5-cm segments and stretching the tube over a three-dimensional conical mold. The PCL scaffold is heat-treated in about 58° C. water bath for one second to partially melt the PCL fibers, effectively increasing the mechanical properties of the conduit and solidifying the funnel shape. For example, FIG. 12A shows a photograph of an embodiment of the presently disclosed conical conduit 200 having a 1.5-mm proximal diameter, 0.5-mm distal diameter, and 10-mm length. Further, FIG. 12B shows a photograph of an example of a positive conical conduit mold 205 with 1.5-mm proximal diameter, 0.5-mm distal diameter, and 15-mm length taper.

The crimped surface of the conduit is achieved by compressing the conduit into a smaller tube while soaked in ethanol before drying. The extensions to the conduit are achieved by leaving a portion of the PCL scaffold tube that is not stretched over the three-dimensional conical mold, thereby creating an appendage to the conduit that is of constant diameter.

The formation of the conical shape and its associated surface crimping and extensions, however, may be obtained through alternative methods, including but not limited to, electrospinning directly onto a mold with the desired structure, or electrospinning onto an isolated charged point with a circular diameter to create a conical fiber shape.

1.3.3 Synthesis of Interpenetrating Network (IPN) Gel

The interpenetrating network (IPN) gel filling includes a nanofiber hydrogel composite (NHC) and chondroitin sulfate proteoglycans (CSPGs). The NHC includes acrylated hyaluronic acid (HA-Ac), cryomilled functionalized PCL fiber fragments, and thiolated poly(ethylene glycol) (PEG-SH).

HA-Ac is either purchased directly from commercial manufacturers or synthesized from hyaluronic acid and glycidyl acrylate. In one embodiment, HA-Ac is produced by creating a 1% w/v solution of HA in phosphate-buffered saline and adding 3.25 mL of glycidyl acrylate to 100 mL of 1% HA solution. The entire solution is shaken at 200 RPM at 37° C. for 16 hours. The solution is dripped into 1 L of 100% ethanol, and the precipitated HA-Ac is collected and washed with 100% ethanol three times before drying in the hood. Synthesis methods for HA-Ac include, but are not limited, to this process.

In one embodiment, 5000 MW PEG-SH is manufactured or purchased commercially. Synthesis methods, however, are available to create PEG-SH or alternate cross-linkers. PCL fiber fragments are produced by electrospinning a PCL nanofiber sheet before surface functionalization and cryomilling. Li et al., 2020. In one embodiment, parameters include spinning a 16 wt % solution of PCL in an 85:15 mixture of 45,000:80,000 MW PCL dissolved in a 9:1 organic solvent of DCM:DMF. Electrospinning parameters include spinning at 16 kV at a distance of 16 cm from a steel drum rotating at 750 RPM. Following the electrospinning, the PCL sheet is cut into 100 cm 2 sheets and plasma treated for 30 minutes. The resulting carboxyl groups on the PCL surface are reacted with N-(3-Dimethylaminopropyl)-N′-ethylcarbodiimide hydrochloride, N-hydroxysuccinimide, and N-(2-Aminoethyl)maleimide trifluoroacetate salt in a 1:10:25:5 molar ratio. The functionalized PCL fiber is then cryomilled by freezing in liquid nitrogen and milling at 6 cycles for 1-minute milling periods and 2-minute cool-down cycles. The resulting cryomilled fibers are filtered through a 100-μm filter and lyophilized before use.

In one embodiment, CSPGs are acquired commercially from biological chicken sources. Additional sources of CSPGs and other inhibitors of axonal growth, however, also can be used in place of these CSPGs.

The overall IPN gel is synthesized by physically mixing HA-Ac, PEG-SH, and PCL fiber fragments to form the NHC. In one implementation, the NHC formulation contains 7 mg/mL HA-Ac, 7 mg/mL PEG-SH, and 8 mg/mL of PCL fibers. The NHC is mixed with CSPGs at a concentration of 200 ρg/mL. Alternative formulations, however, include adjusting the concentrations of all the listed above components or blends, analogs, derivatives, modifications, and mixtures thereof.

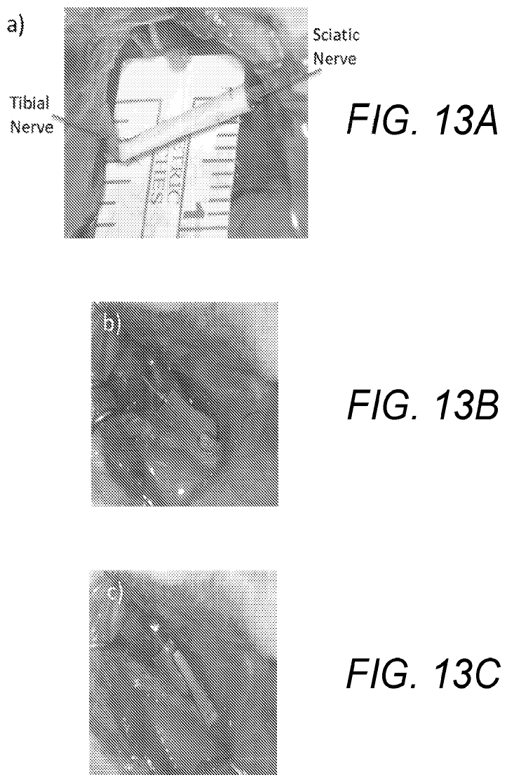

Referring now to FIG. 13A, FIG. 13B, and FIG. 13C, a representative embodiment of the presently disclosed conical conduit is provided for connecting a severed rat sciatic nerve stump with its tibial branch. For example, FIG. 13A shows a photograph of an example of the presently disclosed conical conduit having a 1.5-mm proximal diameter, 0.5-mm distal diameter, and 10-mm length connecting a severed rat sciatic nerve stump and tibial nerve target. FIG. 13B shows a photograph of an example of the size mismatch of a severed sciatic nerve and tibial branch. FIG. 13C shows a photograph of an example of the implementation of the presently disclosed conical conduit that is micro-sutured to the sciatic nerve and the tibial nerve.

### Example 2

### Effect of CSPG-Nanofiber Hydrogel-Filled Funnel Conduit on TMR Outcomes

2.1 Experimental Design

A rat model of TMR was developed to assess the effectiveness of the funnel conduit in guiding regenerating axons across a size-mismatched nerve coaptation, preventing coaptation site neuroma formation, and facilitating re-innervation of the target muscle. In Sprague-Dawley rats, the sciatic nerve was transected and the large caliber proximal nerve was coapted to a single fascicle supplying the lateral gastrocnemius muscle to model a size-mismatched nerve repair that occurs in TMR and other clinical scenarios. Five separate experimental groups were used. All groups underwent nerve transection except for the positive control group, which underwent sham surgery. Two groups underwent repair using a funnel-shaped conduit with the following in the lumen: (1) chondroitin sulfate proteoglygan (CSPG)-incorporated nanofiber-hydrogel composite (NHC); or (2) NHC without CSPG. A group modeling TMR as it is currently performed underwent suture repair without a conduit. The negative control group underwent sciatic nerve transection without repair.

All materials used and methods performed were done so in accordance with protocols approved by the Johns Hopkins University Animal Care and Use Committee and federal guidelines from the National Institutes of Health and the United States Department of Agriculture. The rats were cared for in a double-barrier animal facility in standard cages with continuous fresh air and ad libitum water and food. Rats were maintained for 12 weeks (84 days) post-operation, as established by previous studies on peripheral nerve injury and recovery. Giimus et al., 2015.

At 84 days post-operation, four rats in each experimental group underwent retrograde labelling of the tibial nerve with fluorogold using previously established protocols to visualize motor neurons in the spinal cord that regenerated axons beyond the repair site. In brief, the rats were first anesthetized with isofluorane before a small incision was made, distal to the popliteal fossa, to identify the lateral gastrocnemius branch of the tibial nerve. The branch was transected 2 mm distal to the distal coaptation site. The transected proximal end was placed in a silicone tube containing a 3% fluorogold solution, and the nerve was allowed to rest for 1 hour in the dark to allow uptake of the tracer. The silicone tube and excess solution were then removed, the incision was closed, and the animal was allowed to recover. After allowing time for retrograde transport of the dye, two days later (86 days post-operation) the rats were anesthetized and transcardially perfused with phosphate-buffered saline and 4% paraformaldehyde (PFA). Spinal cord (T11-12) and dorsal root ganglia samples were harvested, post-fixed in 4% PFA overnight, and cryoprotected in 30% sucrose solution for 24 hours. Gastrocnemius muscle and the injury site nerve also were extracted.

The remaining rats were sacrificed at 84 days post-operation and the tissue samples from the spinal cord, dorsal root ganglia, and nerve injury site were harvested and analyzed according to previously established protocols. All tissue samples were embedded in optimal cutting temperature (OCT) tissue freezing medium before being frozen in liquid nitrogen. Spinal cord sections were then longitudinally sectioned at 50-μm thick sections at −20° C. before mounting onto glass slides. The slides were left to dry for 24 hours at 25° C. before either being frozen at −20° C. for preservation or immunostained. For immunohistochemical (IHC) staining, the samples were first incubated with a 4% blocking solution before applying the primary antibody. The samples were left to incubate overnight at 4° C. before being washed and having the secondary antibody applied for 2 hours. Following secondary antibody application, excess solution was aspirated before the slides were stored at 4° C. or immediately imaged under the fluorescent microscope.

Longitudinal spinal cord and nerve sections were stained for TUJ1 to characterize the orderliness of axonal regeneration across the coaptation site and evaluate for neuroma formation. Muscle sections were stained for laminin surrounding the myofibers to allow for quantification of myofiber cross-sectional area. Sections were also stained with DAPI to visualize cell nuclei presence.

2.2 Results and Discussion

2.2.1 General Outcomes

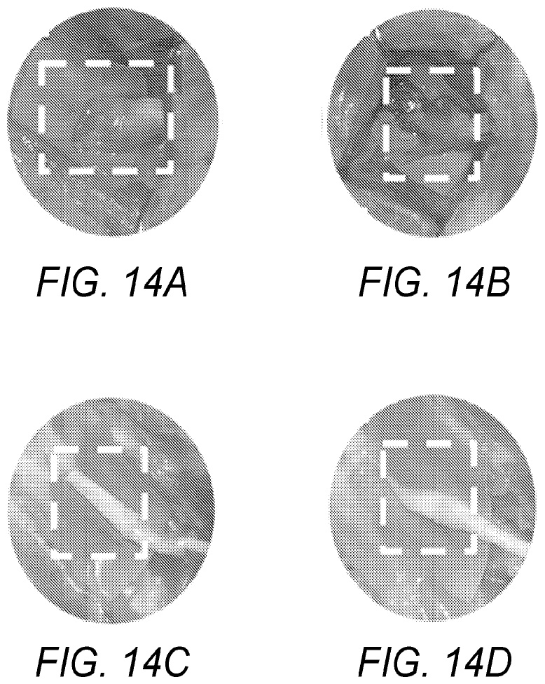

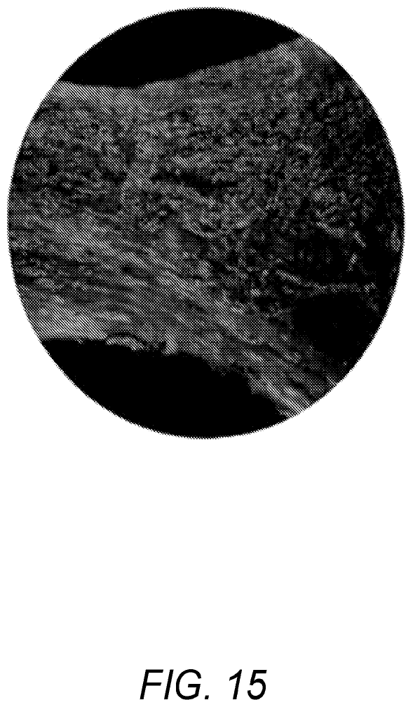

Referring now to the photographs shown FIG. 14A, FIG. 14B, FIG. 14C, FIG. 14D, and FIG. 15, FIG. 14A shows an example of a proximal sciatic nerve stump (right) coapted to distal tibial nerve fascicle supplying the lateral gastrocnemius muscle, modeling the coaptation size-mismatch that occurs in TMR clinically. FIG. 14B shows an example of a neuroma formation at the surgical site 12-weeks post-TMR. FIG. 14C shows an example of a CSPG-conduit implanted within a size-mismatched coaptation site. FIG. 14D shows an example of a tapered reinnervation without gross evidence of the neuroma formation at the surgical site 12-weeks post TMR with implanted CSPG-conduit. FIG. 15 shows an example of how TUJ1-staining of longitudinal nerve sections demonstrates orderly, tapered axonal growth without axonal escape and neuroma formation in the CSPG-Conduit group.

Qualitative gross evaluation of the coaptation site 84 days after size-mismatched nerve repair without use of a conduit (TMR, see FIG. 14A) revealed the size-mismatch between the sciatic nerve and tibial branch (see FIG. 14A) demonstrated obvious neuroma formation at the coaptation site (see FIG. 14B). For the CSPG-Conduit and Funnel Conduit groups, the device was implanted immediately following nerve transection (see FIG. 14C). In the CSPG-Conduit group, qualitative gross evaluation of the coaptation site 84 days post implantation demonstrated evenly tapered neural tissue with no evidence of neuroma formation (see FIG. 14D). Immunofluorescence staining with TUJ1 of the longitudinal nerve sections demonstrated orderly, tapered axonal growth across the coaptation site and into the distal nerve stump, without axonal escape and neuroma formation (see FIG. 15).

These results support the hypothesis that the use of a funnel conduit with the CSPG-NHC within the lumen to bridge a size-mismatched nerve coaptation is effective guiding axonal regeneration across a size-mismatched nerve repair and preventing neuroma formation that occurs when TMR is performed without a tapered conduit. The images support the efficacy of the device in providing both mechanical guidance to prevent axonal escape and chemical inhibition of excessive regeneration that would otherwise overwhelm the capacity of the small caliber distal nerve stump, as the tapered reinnervation exhibited a conical shape and was observed for the CSPG-Conduit group, but not the Funnel Conduit without CSPG group. Future studies will involve analysis of macrophage presence to determine the immunological response to the implant. Further, future studies will analyze the degree of taper between the distal and proximal targets as well as the degree of vascularization in new axonal growth following operations and treatment groups. Without wishing to be bound to any one particular theory, it is thought that the CSPG-Conduit group will demonstrate a significantly increased degree of vascularization and a decreased immunological response as compared to other experimental groups. Future studies will characterize regeneration of the various sensory axonal subtypes implicated in painful neuroma formation, expression of pain-associated genes, and behavioral testing to evaluate pain response.

2.2.2 Gastrocnemius Muscle Weight

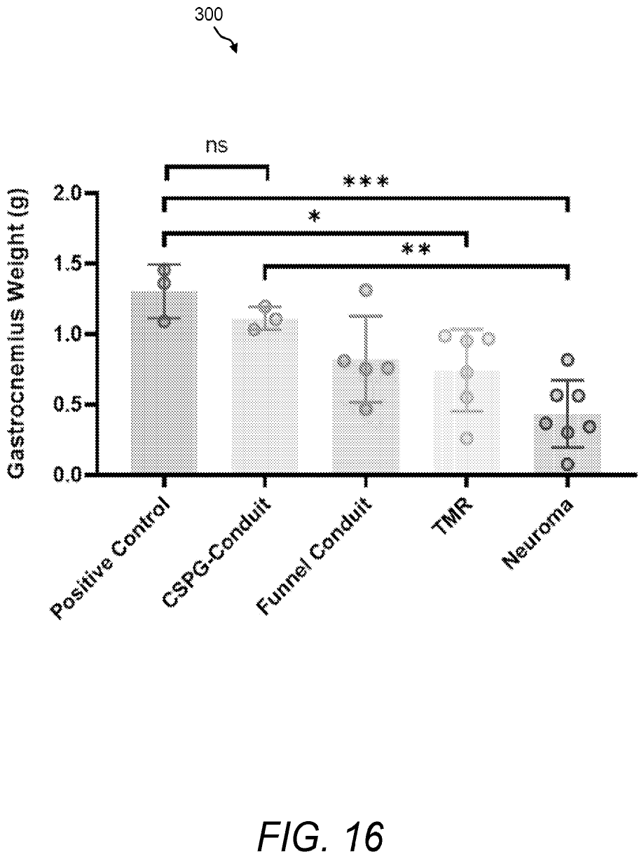

The gastrocnemius muscle of the Sprague-Dawley rats was harvested immediately after sacrifice at 84 days, and then massed. Gastrocnemius muscle weight is a validated measure of the speed and magnitude of muscle reinnervation, as progressive denervation-induced muscle atrophy with loss of muscle weight occurs in the absence or reinnervation. For example, FIG. 16 shows an example of a plot 300 showing that the greater mean gastrocnemius mass indicates greater functional nerve recovery obtained in the CSPG-conduit group compared with the negative control (neuroma group). No significant difference between the positive control and the CSPG-Conduit group was observed. The bars of plot 300 represent mean±SD (ns=not significant, *p≤0.05, **p≤0.01, ***p≤0.001).

The weights of the lateral gastrocnemius muscles collected from all experimental groups were compared. Statistical analyses were performed using ordinary one-way ANOVA with Tukey post hoc testing. The CSPG-Conduit group demonstrated the largest average mass (1.112±0.081 g) aside from the positive control group (1.304±0.191 g). In contrast, the average muscle masses for the NHC-only funnel conduit group was 0.8214±0.306 g, followed by 0.742±0.291 g for the TMR group, and 0.434±0.238 g for the negative control (neuroma) group. The general trend of average gastrocnemius mass followed the hypothesized trend that the TMR (without conduit) group would exhibit greater muscle mass than negative control group that did not undergo repair; and the funnel conduit without CSPG treatment would exhibit greater mass than the TMR group. Greater mean gastrocnemius mass correlates to greater muscle reinnervation and functional recovery. Significant greater muscle weight was observed in the positive control (uninjured) in comparison to the TMR and negative control groups, whereas no significant difference was observed between the positive control and CSPG-Conduit groups, supporting the efficacy of the CSPG-Conduit for functional nerve recovery. Further, significant differences were observed between the CSPG-Conduit and Neuroma groups.

It should be noted that there was no statistical significance (p=0.2751) in the average gastrocnemius masses between the CSPG-Conduit and the TMR group despite a nearly 50% greater average mass in the former compared to the latter. This lack of significance is likely as a result of the smaller sample size (n=3 for CSPG-Conduit group, n=6 for TMR group) which will be addressed in subsequent, larger-scale studies.

### Example 3

### Evaluation of the Behavioral Response to Mechanical Stimuli of the Neuroma

To evaluate the behavioral response to mechanical stimuli of the neuroma, a 15-G nylon monofilament was used to mechanically stimulate the area above the coaptation site. The nylon monofilament was applied to the area 10 times; each time for 2-3 seconds with a 1- to 2-minute interval in between trials. Responses were graded on a scale of 0-2 with a grade 0 being no response to the stimuli, a grade 1 response was defined as a slow withdrawal of the hind paw, and the grade 2 response consisted of a rapid withdrawal of the hind paw, licking of the area, shaking of the limb, or vocalization. The behavioral response score to evaluate neuroma pain was defined as the sum of responses for 10 trials, ranging from 0 to 20 with higher scores indicating greater pain. All behavioral tests were performed by a blinded examiner.

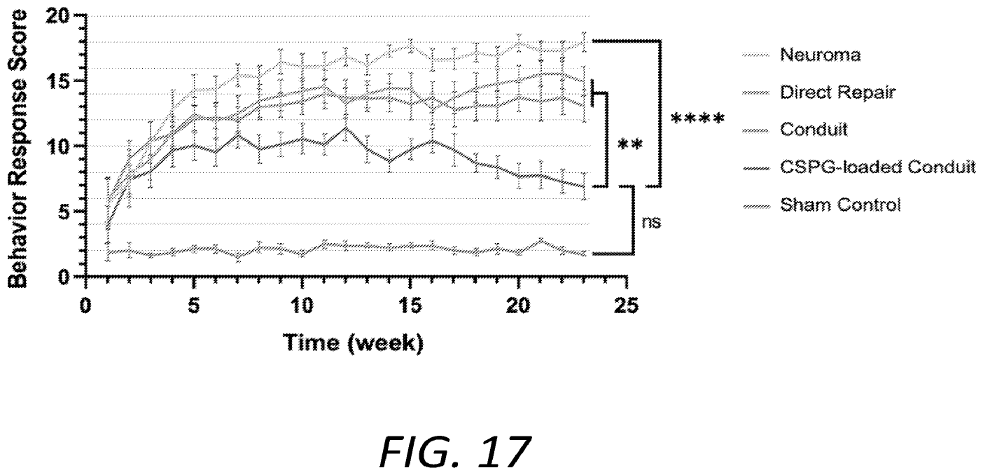

Referring now to FIG. 17, by week 23, behavioral responses to mechanical stimulation of the coaptation site were significantly lower in animals receiving CSPG-loaded Conduit treatment as compared to those in the funnel Conduit (p<0.01), the Direct Repair (p<0.01), and the Neuroma groups (p<0.0001), demonstrating successful prevention of neuroma formation at the coaptation site. This decline in pain responses in the CSPG-loaded Conduit group is particularly observed after Week 12 when the majority of nerves have fully regenerated and the pain inherent to regeneration is diminished. In contrast, the Neuroma group pain scores continue to increase beyond Week 12, nearing the maximal pain score of 20. Direct Repair animals consistently exhibited greater pain responses than CSPG-loaded Conduit animals even in the early regeneration period between Weeks 4 and 10, demonstrating the utility of the CSPG-loaded Conduit in comparison to nerve Direct Repair alone. The positive control (sham surgery) group consistently maintained low response scores throughout the testing period and pain scores between the CSPG-loaded Conduit and positive control (sham surgery) are no longer statistically significant at Week 23 (p=0.1347). Conduit and Direct Repair groups exhibited similar pain responses throughout the testing period. At Week 23, Conduit group pain scores were significantly lower than Neuroma scores (p<0.01), indicating a benefit in using the conduit alone.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Cravioto, H., & Battista, A. (1981). Clinical and ultrastructural study of painful neuroma. Neurosurgery, 8(2), 181-190.
Weng, W., Zhao, B., Lin, D., Gao, W., Li, Z., & Yan, H. (2016). Significance of alpha smooth muscle actin expression in traumatic painful neuromas: a pilot study in rats. Scientific reports, 6, 23828.
Lu, C., Sun, X., Wang, C., et al. (2018). Mechanisms and treatment of painful neuromas. Reviews in the Neurosciences, 29(5), pp. 557-566. Retrieved 31 Oct. 2019, from doi:10.1515/revneuro-2017-0077
Dellon, A. L., & Mackinnon, S. E. (1986). Treatment of the painful neuroma by neuroma resection and muscle implantation. Plastic and reconstructive surgery, 77(3), 427-438.
Wood, W. (1829). Observations on neuroma, with cases and histories of the disease. Transactions. Medico-Chirurgical Society of Edinburgh, 3(Pt 2), 367.
Morton's neuroma surgery, stump neuroma, Wallerian degeneration. (2018, September 18). Retrieved from https://www.mortonsneuroma.com/blog/mortons-neuroma-surgery-stump-neuroma-walleriandegeneration/.
Bowen J B, Wee C E, Kalik J, Valerio I L. Targeted Muscle Reinnervation to Improve Pain, Prosthetic Tolerance, and Bioprosthetic Outcomes in the Amputee. Adv Wound Care (New Rochelle). 2017 Aug. 1; 6(8):261-7. Epub 2017/08/24.
Eberlin K R, Ducic I. Surgical Algorithm for Neuroma Management: A Changing Treatment Paradigm. Plast Reconstr Surg Glob Open. 2018 October;6(10):e1952. Epub 2018/12/12.
Neuroma and Phantom Limb Pain: Institute for Advanced Reconstruction. (2017, September 1). Retrieved from https://www.advancedreconstruction.com/nerve-surgery/neuroma-and-phantom-limb-pain/.
Kuiken, T. A., Barlow, A. K., Hargrove, L., & Dumanian, G. A. (2017). Targeted muscle reinnervation for the upper and lower extremity. Techniques in orthopaedics (Rockville, Md.), 32(2), 109.
Souza, J. M., Cheesborough, J. E., Ko, J. H., Cho, M. S., Kuiken, T. A., & Dumanian, G. A. (2014). Targeted muscle reinnervation: a novel approach to postamputation neuroma pain. Clinical Orthopaedics and Related Research®, 472(10), 2984-2990.
Dumanian, G. A., Potter, B. K., Mioton, L. M., Ko, J. H., Cheesborough, J. E., Souza, J. M., . . . & Kuiken, T. A. (2019). Targeted muscle reinnervation treats neuroma and phantom pain in major limb amputees: a randomized clinical trial. Annals of surgery, 270(2), 238-246.
Gart, M. S., Souza, J. M., & Dumanian, G. A. (2015). Targeted muscle reinnervation in the upper extremity amputee: a technical roadmap. The Journal of hand surgery, 40(9), 1877-1888.
Bergmeister, K. D., Aman, M., Muceli, S., Vujaklija, I., Manzano-Szalai, K., Unger, E., Byrne, R. A., Scheinecker, C., Riedl, O., Salminger, S., Frommlet, F., Borschel, G. H., Farina, D., & Aszmann, O. C. (2019). Peripheral nerve transfers change target muscle structure and function. Science advances, 5(1), eaau2956. https://doi.org/10.1126/sciadv.aau2956
Mackinnon, S. E. (1989). New directions in peripheral nerve surgery. Annals of plastic surgery, 22(3), 257-273.
U.S. Pat. No. 10,405,963 for Method of producing a 3D subject specific biomimetic nerve conduit, to McAlpine et al., issued Sep. 10, 2019.
U.S. Pat. No. 8,728,817 for Compositions and methods for making and using laminin nanofibers, to Ogle et al., issued May 20, 2014.
U.S. Patent Application Publication No. 2020/0030237A1, for Localized Jan. 30, 2020.
U.S. Pat. No. 9,925,695, for Poly(lactic-co-glycolic acid (PLGA) composites with magnesium wires enhanced networking of primary neurons, to Liu et al., issued Mar. 27, 2018.
Langhals, N. B., Cederna, P. S., & Urbanchek, M. G. (2016). U.S. Pat. No. 9,352,146. Washington, DC: U.S. Patent and Trademark Office.
Okuda, T., Ishida, O., Fujimoto, Y., Tanaka, N., Inoue, A., Nakata, Y., & Ochi, M. (2006). The autotomy relief effect of a silicone tube covering the proximal nerve stump. Journal of orthopaedic research, 24(7), 1427-1437.
Li, X., Cho, B., Martin, R., Seu, M., Zhang, C., Zhou, Z., . . . & Mao, H. Q. (2019). Nanofiber-hydrogel composite-mediated angiogenesis for soft tissue reconstruction. Science translational medicine, 11(490), eaau6210.
Sarhane, K. A., Ibrahim, Z., Martin, R., Krick, K., Cashman, C. R., Tuffaha, S. H., . . . & Brandacher, G. (2019). Macroporous nanofiber wraps promote axonal regeneration and functional recovery in nerve repair by limiting fibrosis. Acta biomaterialia, 88, 332-345.
Al-Enizi, A. M., Zagho, M. M., & Elzatahry, A. A. (2018). Polymer-Based Electrospun Nanofibers for Biomedical Applications. Nanomaterials (Basel, Switzerland), 8(4), 259. https://doi.org/10.3390/nano8040259
Painter, P. C., & Coleman, M. M. (2008). Essentials of polymer science and engineering. DEStech Publications, Inc.
Li, X., Zhang, C., Haggerty, A. E., Yan, J., Lan, M., Seu, M., . . . & Mao H. Q. (2020). The effect of a nanofiber-hydrogel composite on neural tissue repair and regeneration in the contused spinal cord. Biomaterials, 119978.
Lemons, M. L., Howland, D. R., & Anderson, D. K. (1999). Chondroitin sulfate proteoglycan immunoreactivity increases following spinal cord injury and transplantation. Experimental neurology, 160(1), 51-65.
Snow, D. M., Steindler, D. A., & Silver, J. (1990). Molecular and cellular characterization of the glial roof plate of the spinal cord and optic tectum: a possible role for a proteoglycan in the development of an axon barrier. Developmental biology 138(2), 359-376.
Batt, J. A., & Bain, J. R. (2013). Tibial nerve transection—a standardized model for denervation-induced skeletal muscle atrophy in mice. Journal of visualized experiments: JoVE, (81), e50657.
Gumus B., Kuyucu, E., Erbas, O., Kazimoglu, C., Oltulu, F., & Bora, O. A. (2015). Effect of oxytocin administration on nerve recovery in the rat sciatic nerve damage model. Journal of orthopaedic surgery and research, 10, 161.
Gart, M. S., Souza, J. M., Dumanian, G. A. (2015). Targeted Muscle Reinnervation in the Upper Extremity Amputee: A Technical Roadmap. The Journal of hand surgery. 40(9): 1877-1888.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nerve conduit comprising a tubular body having a proximal aperture and a distal aperture, wherein the proximal aperture has a diameter greater than a diameter of the distal aperture.

2. The nerve conduit of claim 1, wherein the tubular body has a shape selected from a conical shape, a concave shape, and a convex shape.

3. The nerve conduit of claim 1 or claim 2, wherein the tubular body has a right conical shape.

4. The nerve conduit of claim 3, wherein the right circular cone has a linear increase in diameter size from the distal aperture to the proximal aperture.

5. The nerve conduit of any one of claims 1-4, wherein the tubular body has a conical shape that tapers in decreasing diameter from the proximal aperture to the distal aperture.

6. The nerve conduit of any one of claims 1-5, wherein the tubular body has a conical shape having a taper range of about 1° to about 89°.

7. The nerve conduit of any one of claims 1-6, wherein the tubular body comprises a lumen comprising a hydrogel.

8. The nerve conduit of claim 7, wherein the hydrogel further comprises one or more of a fibrin-, a collagen-, a tissue matrix-derived hydrogel, or combinations thereof.

9. The nerve conduit of claim 7, wherein the tubular body or hydrogel comprises one or more agents for inhibiting axonal growth, polarizing macrophages to the pro-regenerative phenotype, supporting angiogenesis, and combinations thereof.

10. The nerve conduit of claim 9, wherein the tubular body or hydrogel comprises one or more components selected from a nanofiber hydrogel composite (NHC) and one or more bioactive agents that inhibit axonal outgrowth.

11. The nerve conduit of claim 10, wherein the one or more bioactive agents that inhibit axonal outgrowth are selected from semaphorin, a myelin-associated glycoprotein, and one or more chondroitin sulfate proteoglycans (CSPGs).

12. The nerve conduit of claim 11, wherein the NHC comprises functionalized poly(ε-caprolactone) (PCL) fiber fragments distributed in and covalently conjugated to a hydrogel network formed by reacting acrylated hyaluronic acid (HA) with thiolated poly(ethylene glycol) (PEG-SH).

13. The nerve conduit of claim 11, wherein the hydrogel comprises NHC and one or more chondroitin sulfate proteoglycans (CSPGs).

14. The nerve conduit of claim 7, wherein the hydrogel has an overall stiffness (storage modulus, G′) ranging from about 50 Pa to about 500 Pa.

15. The nerve conduit of claim 7, wherein the hydrogel comprises an interpenetrating network (IPN).

16. The nerve conduit of claim 1, wherein the tubular body comprises a wall comprising a nanofiber diameter and pore size sufficient to allow diffusion of nutrients while preventing inflammatory macrophage infiltration.

17. The nerve conduit of claim 16, wherein the tubular body comprises a nanofiber mesh wall having a substantially uniform thickness ranging from about 50 μm to about 500 μm and with a pore size of less than about 10 μm.

18. The nerve conduit of claim 17, wherein the nanofiber mesh wall comprises randomly oriented nanofibers having a diameter ranging from about 100 nm to about 2 μm.

19. The nerve conduit of claim 17 or claim 18, wherein the nanofiber mesh wall comprises a synthetic material selected from poly(ε-caprolactone) (PCL), copolymers of ε-caprolactam and hexamethylendiaminadipate, polyglycolic acid (PGA), poly(lactic acid) (PLA), poly (1-lactic acid) (PLLA), copolymers of PLA and PGA, poly(lactic-co-glycolic acid) (PLGA), poly(vinyl acetate) (PVA), poly(ethylene-co-vinyl acetate) (PEVA), poly(ethylene glycol) (PEG), polyurethanes (PU), poly(ethylene oxide) (PEO), poly(vinyl pyrrolidone) (PVP), poly(ethylene terephthalate) (PET), poly(glycerol sebacate) (PGS), polydioxanone (PDO), polyphosphazenes (PPHOs), polyhydroxyalkanoates (PHA), polyhydroxybutyrates (PHB), polyhydroxyvalerate (PHV), polyhydroxyhexanoate (PHH), and polyhydroxyoctanoate (PHO), as well as co-polymers, blends, analogs, derivatives, modifications, and mixtures thereof.

20. The nerve conduit of claim 17 or claim 18, wherein the nanofiber mesh wall comprises a natural material selected from hyaluronic acid (HA), silk, keratin, collagen, gelatin, fibrinogen, elastin, actin, myosin, cellulose, amylose, dextran, chitin, glycosaminoglycans (GAG), deoxyribonucleic acids (DNA), ribonucleic acids (RNA), chitin, chitosan (CS), alginate, as well as co-polymers, blends, analogs, derivatives, modifications, and mixtures thereof.

21. The nerve conduit of claim 1, wherein the tubular body comprises a smooth wall, a crimped wall, or combinations thereof.

22. The nerve conduit of claim 21, wherein the crimped wall comprises one or more ridges characterized by a kink-resistance of up to a 90° bend and a length adjustability of less than or equal to 100% of an initial length of the conduit.

23. The nerve conduit of claim 21, wherein the crimped wall comprises a crimp pattern having a plurality of crests and troughs.

24. The nerve conduit of claim 21, wherein the crimped wall has a thickness (h), a width (w), a first diameter (d₁), a second diameter (d₂), a first length (l₁), a second length (l₂), and a third length (l₃),
wherein h has a range of about 0.1 mm to 5.0 mm, with a ratio of w to h having a range of about 0.1 to about 10;
wherein d₁ has a range of about 0.5 mm to about 25 mm, d₂ has a range of about mm to about 10 mm, and d₂<d₁; and
wherein l₁ has a range of about 0 mm to about 10 mm, l₂ has a range of about 2 mm to about 50 mm, and l₃ has a range of about 0 mm to about 10 mm.

25. The nerve conduit of claim 24, wherein the thickness (h) is about 0.5 mm to about 2 mm, the width (w) is about 0.5 mm to about 1 mm, the first diameter (d₁) is about 5 mm to about 10 mm, the second diameter (d₂) is about 1 mm to about 4 mm, the first length (l₁) is about 2 mm to about 5 mm, the second length (l₂) is about 5 mm to about 20 mm, and the third thickness (l₃) is about 2 mm to about 5 mm.

26. The nerve conduit of claim 1, wherein the tubular body further comprises one or more of a proximal extension, a distal extension, and combinations thereof.

27. The nerve conduit of claim 26, wherein the one or more extensions are adapted for suturing.

28. The nerve conduit of claim 26, wherein the one or more extensions has one or more dimensions ranging in diameter from about 50 μm to about 25 mm and in length from about 0 mm to about 50 mm.

29. The nerve conduit of claim 1, wherein the proximal aperture and the distal aperture have a diameter ranging from about 50 μm to about 25 mm, provided that the diameter of the proximal aperture is greater than the diameter of the distal aperture.

30. The nerve conduit of claim 29, wherein the proximal aperture has a diameter of about 1.5 mm and the distal aperture has a diameter of about 0.5 mm.

31. The nerve conduit of claim 1, wherein the tubular body has a length ranging from about 5 mm to about 50 mm.

32. The nerve conduit of claim 31, wherein the tubular body has a length of about 10 mm.

33. The nerve conduit of claim 1, wherein the tubular body has one or more dimensions comprising a 1.5-mm proximal aperture diameter, a 0.5-mm distal aperture diameter, and a 10-mm length.

34. A method for treating or repairing nerve injury in a subject in need of treatment thereof, the method comprising:
providing a nerve conduit of any one of claims 1-33; and
contacting a large caliber injured nerve with the proximal aperture of the nerve conduit and contacting a small caliber sensory nerve with the distal aperture of the nerve conduit.

35. The method of claim 34, wherein the nerve comprises a peripheral nerve.

36. The method of claim 35, wherein the nerve conduit comprises a conical nerve conduit comprising a CSPG-containing hydrogel and the method comprises targeted muscle reinnervation (TMR) or preventing neuroma in a subject having an injured nerve.

37. The method of claim 36, wherein the injured nerve is a result of a limb amputation.

38. The method of claim 34, wherein the method comprises targeted sensory reinnervation (TSR) for painful neuroma prevention and/or treatment, wherein the large caliber injured nerve is coapted to the small caliber sensory nerve.

39. The method of claim 34, wherein the method comprises targeted muscle reinnervation (TMR) for preventing neuroma, wherein the lumen of the nerve conduit comprises hydrogel only or does not include hydrogel.

40. The method of claim 39, wherein the method comprises targeted sensory reinnervation (TSR) for afferent sensory input from a prosthesis.

41. The method of claim 40, wherein the method comprises size-mismatched motor and sensory nerve transfers for motor and sensory functional restoration.

42. The method of claim 34, wherein the method comprising targeted muscle reinnervation (TMR).

43. The method of claim 42, wherein the nerve conduit comprises an empty conduit or a hydrogel-filled conduit without CSPGs.

44. The method of claim 42 or claim 43, wherein the method maximizes the number of motor axons to innerve a target muscle.

45. The method of claim 44, wherein the method improves signal transduction from the target muscle.

46. The method of claim 45, wherein the target muscle is used for efferent signal amplification for prosthesis control.
